(19) United States
(12) Patent Application Publication
(10) Pub. No.: US 2010/0191000 A1
(43) Pub. Date: Jul. 29, 2010

(54) METHOD FOR SYNTHESIZING AN N-UNSUBSTITUTED OR N-SUBSTITUTED AZIRIDINE

(75) Inventors: Johann-Peter Melder, Bohl-Iggelheim (DE); Martin Ernst, Heidelberg (DE); Till Gerlach, Ludwigshafen (DE); Ekkehard Schwab, Neustadt (DE); Csaba Varszegi, Genk (BE); Bert Sels, Balen (BE); Dirk de Vos, Holsbeek (BE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/665,509

(22) PCT Filed: Jun. 18, 2008
(86) PCT No.: PCT/EP08/57702

§ 371 Date: Dec. 18, 2009

(30) Foreign Application Priority Data
Jun. 20, 2007 (EP) 07110690.0
Jun. 5, 2008 (EP) 08157633.2

(51) Int. Cl. C07D 203/08; C25B 3/02

(52) U.S. Cl. 548/969; 548/954; 205/431

(57) ABSTRACT

Process for preparing an N-unsubstituted or N-substituted aziridine of the formula

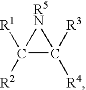

which comprises reacting an olefin of the formula I

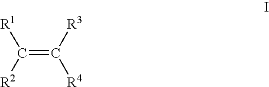

where R¹ to R⁵ are each, independently of one another, hydrogen, a linear or branched alkyl radical having from 1 to 16 carbon atoms, a hydroxyalkyl radical having from 1 to 4 carbon atoms, a cycloalkyl radical having from 5 to 7 carbon atoms, a benzyl or phenyl radical which in each case may be substituted in the o, m or p position of the phenyl radical by methoxy, hydroxy, chlorine or alkyl radicals having from 1 to 4 carbon atoms and the radical R¹ or R² together with the radical R³ or R⁴ may be closed to form a 5- to 12-membered ring or the radicals R¹ and R² may be closed to form a 5- to 12-membered ring, with ammonia or a primary amine of the formula R⁵NH₂ in the presence of iodine or bromine.

The present invention relates to a process for preparing an N-unsubstituted or N-substituted aziridine.

N-unsubstituted aziridines and N-substituted aziridines are important organic intermediates which have a high reactivity and are employed, for example, for preparing polymers and heterocycles.

Aziridine (C₂H₅N) is prepared industrially by epoxidation of ethylene by means of air or oxygen to form ethylene oxide, ring opening of the latter by means of ammonia to give a mixture of monoethanolamine, diethanolamine and triethanolamine, separation of ethanolamine from this mixture, esterification of ethanolamine by means of sulfuric acid to form beta-aminoethylsulfuric acid and cyclization of the product to give aziridine. Here, two mol of sodium hydroxide are used per mol of aziridine, forming one mol of sodium sulfate (H. J. Arpe, Industrielie Organische Chemie, 6th edition 2007, Wiley-VCH-Verlag, pages 158 to 160 and 172 to 174). Substituted aziridines can also be obtained in a similar way.

Disadvantages of these processes are the numerous process steps and the stoichiometric formation of a salt.

It is known that aziridines substituted on the nitrogen by p-toluenesulfonyl radicals can be prepared by reaction of olefins with chloramine T (obtainable from p-toluenesulfonamide and sodium hypochlorite), potassium carbonate, silicon dioxide and catalytic amounts of iodine (S. Minakata et al., Angew, Chem. int. Ed. 2004, 43, pages 79 to 81).

Disadvantages here are that the aziridine nitrogen is introduced by means of the chloramine T which can be prepared in a multistage synthesis and stoichiometric amounts of chloramine T are therefore required. This also means that only N-substituted aziridines can be obtained and stoichiometric amounts of sodium chloride are formed.

It is also known that aziridines substituted on the nitrogen by p-toluenesulfonyl radicals can be synthesized by reaction of olefins with p-toluenesulfonamide and tert-butyl hypolodite prepared in situ from tert-butyl hypochlorite and sodium iodide (S. Minakata et al., Chem. Commun. 2006, pages 3337 to 3339 and JP-A-2007 055958).

This method has disadvantages similar to those of the above-described process using chloramine T.

It is therefore an object of the invention to overcome the disadvantages of the prior art and to discover a process which makes it possible to prepare N-unsubstituted and N-substituted aziridines from olefins in few reaction steps, if possible with no or only little formation of salts.

According to the invention, it has been recognized that it would be significantly more advantageous to introduce the aziridine nitrogen by means of ammonia or a primary amine.

We have accordingly found a process for preparing an N-unsubstituted aziridine of the formula II

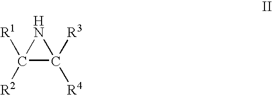

which comprises reacting an olefin of the formula I

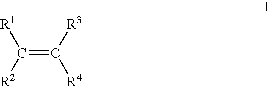

where R¹ to R⁴ are each, independently of one another, hydrogen, a linear or branched alkyl radical having from 1 to 16 carbon atoms, a hydroxyalkyl radical having from 1 to 4 carbon atoms, a cycloalkyl radical having from 5 to 7 carbon atoms, a benzyl or phenyl radical which in each case may be substituted in the o, m or p position of the phenyl radical by methoxy, hydroxy, chlorine or alkyl radicals having from 1 to 4 carbon atoms and the radical R¹ or R² together with the radical R³ or R⁴ may be closed to form a 5- to 12-membered ring or the radicals R¹ and R² may be closed to form a 5- to 12-membered ring, with ammonia in the presence of iodine or bromine.

Furthermore, we have found a process for preparing an N-unsubstituted aziridine of the formula II, which comprises reacting an olefin of the formula I with ammonia in the presence of an iodide and an oxidant which is able to oxidize the iodide to iodine.

Furthermore, we have found a process for preparing an N-unsubstituted aziridine of the formula II, which comprises reacting an olefin of the formula I with ammonia in the presence of a bromide and an oxidant which is able to oxidize the bromide to bromine.

Furthermore, we have found a process for preparing an N-substituted aziridine of the formula III,

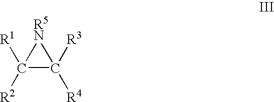

which comprises reacting an olefin of the formula I

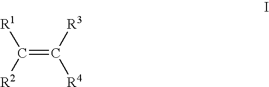

where R¹ to R⁴ are each, independently of one another, hydrogen and R¹ to R⁵ are each, independently of one another, linear or branched alkyl radicals having from 1 to 16 carbon atoms, hydroxyalkyl radicals having from 1 to 4 carbon atoms, cycloalkyl radicals having from 5 to 7 carbon atoms, benzyl radicals and phenyl radicals which may in each case be substituted in the o, m or p position of the phenyl radical by methoxy, hydroxy, chlorine or alkyl radicals having from 1 to 4 carbon atoms, and the radicals R¹ or R² can be closed with the radicals R³ or R⁴ to form a 5- to 12-membered ring or the radicals R¹ and R² can be closed to form a 5- to 12-membered ring, with a primary amine of the formula R⁵NH₂ in the presence of iodine or bromine, where the concentration of the primary amine (R⁵NH₂) in the reaction mixture is less than or equal to 1.1 molar (≦1.1 M).

Furthermore, we have found a process for preparing an aziridine of the formula which comprises reacting an olefin of the formula I with a primary amine of the formula R⁵NH₂ in the presence of an iodide and an oxidant which is able to oxidize the iodide to iodine, where the concentration of the primary amine (R⁵NH₂) in the reaction mixture is less than or equal to 1.1 molar (≦1.1 M).

Furthermore, we have found a process for preparing an aziridine of the formula III, which comprises reacting an olefin of the formula I with a primary amine of the formula R⁵NH₂ in the presence of a bromide and an oxidant which is able to oxidize the bromide to bromine, where the concentration of the primary amine (R⁵NH₂) in the reaction mixture is less than or equal to 1.1 molar (≦1.1 M).

In the process for preparing an N-substituted aziridine of the formula II, the concentration of the ammonia in the reaction mixture at the beginning of the reaction is preferably greater than or equal to 1.2 molar (≧1.2 M), in particular greater than or equal to 1.25 molar (≧1.25 M), e.g. in the range from ≧1.2 to 15 molar, particularly preferably in the range from ≧1.2 to 2 molar.

In the process for preparing an N-substituted aziridine of the formula III, the concentration of the primary amine (R⁵NH₂) in the reaction mixture is preferably less than or equal to 1.0 molar (≦1.0 M). The concentration of the primary amine (R⁵NH₂) in the reaction mixture at the beginning of the reaction is preferably greater than 0.5 molar (>0.5 M), particularly preferably greater than 0.7 molar (>0.7 M), very particularly preferably greater than 0.8 molar (>0.8 M).

According to the invention, it has been recognized that the process for preparing an N-substituted aziridine of the formula III proceeds particularly advantageously, in particular in respect of yield and selectivity, only when an initial concentration of the primary amine (R⁵NH₂) in the reaction mixture is set in the abovementioned ranges (from >0.5 to ≦1.1 M, particularly preferably from >0.8 to ≦0.1 M).

The reaction according to the invention can, for example when using styrene as olefin and ammonia (and water as solvent), be represented by the following reaction equation:

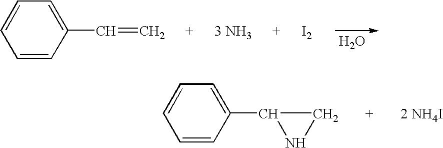

The preferred embodiment of the process using iodides and oxidants can, for example when using styrene as olefin, ammonia, water as solvent, ammonium iodide as iodide and sodium hypochlorite as oxidant, be represented by the following reaction equation:

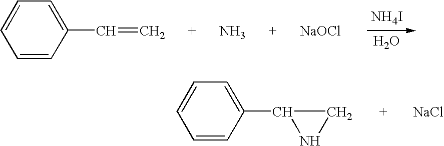

An analogous situation applies when using a primary amine (R⁵NH₂) instead of ammonia.

Examples of radicals R¹ to R⁴ in the olefins of the formula I are as follows: H, methyl, ethyl, n-propyl, isopropyl, n-butyl, isobutyl, n-pentyl, n-hexyl, n-heptyl, n-octyl, n-nonyl, n-decyl, 3-hydroxypropyl, 4-hydroxybutyl, cyclopentyl, cyclohexyl.

Examples of suitable olefins I are ethylene, propylene, i-butene, 1-butene, 2-butene, 1-pentene, 1-hexene, 2-hexene, cyclopentene, methylenecyclopentane, cyclohexene, methylenecyclohexane, 3-hexene, 2-methyl-1-heptene, 1-octene, cyclooctene, 2-octene, 1-dodecene, styrene, alpha-methylstyrene, beta-methylstyrene, p-methylstyrene, p-methoxystyrene, p-hydroxystyrene, m-chlorostyrene, p-chlorostyrene, 2-buten-1-ol, 2-butene-1,4-diol.

Ammonia is preferably used as an aqueous solution which can preferably comprise from 0.1 to 30% by weight of ammonia. The reaction according to the invention can also be carried out in the presence of compounds which are able to liberate ammonia under the reaction conditions.

Examples of radicals R⁵ in the primary amine (R⁵NH₂) are as follows: methyl, ethyl, n-propyl, isopropyl, n-butyl, isobutyl, n-pentyl, n-hexyl, n-heptyl, n-octyl, n-nonyl, n-decyl, cyclopentyl, cyclohexyl.

Particularly preferred primary amines (R⁵NH₂) are methylamine and ethylamine.

The primary amine (R⁵NH₂) is preferably used as aqueous solution. The solution can comprise primary amine up to the saturation solubility. The reaction according to the invention can also be carried out in the presence of compounds which are able to liberate the primary amine under the reaction conditions.

In an embodiment of the process, it is possible to use iodine and/or iodides. Bromine and bromides can also be used instead of iodine and iodides. Iodine and iodides are preferred over bromine and bromides.

Suitable iodides or bromides are alkali metal, alkaline earth metal, ammonium and tetraalkylammonium iodides or alkali metal, alkaline earth metal, ammonium and tetraalkylammonium bromides, where the alkyl radicals in the alkylammonium halides preferably each comprise, independently of one another, from 1 to 5 carbon atoms, and N-haloimides.

Examples of such halides are: ammonium iodide, ammonium bromide, N-bromo-succinimide, N-iodosuccinimide, sodium iodide, sodium bromide, potassium iodide, potassium bromide, magnesium iodide, magnesium bromide, tetramethylammonium iodide, tetramethylammonium bromide; particular preference is given to ammonium iodide and ammonium bromide.

It is also possible to use mixtures of iodides and elemental iodine in place of iodides and mixtures of elemental bromine and bromides in place of bromides.

In the case of mixtures of iodide and iodine, the molar ratio of iodide to iodine can be from 1:0.01 to 0.01:1. The same molar ratio applies to bromides and bromine.

Oxidants used in the processes of the invention are able to oxidize iodides to iodine or bromides to bromine. Suitable oxidants are, for example, oxygen, e.g. in the form of air, hydrogen peroxide, preferably as an aqueous solution, alkyl hydroperoxides such as cumene hydroperoxide, tert-butyl hydroperoxide, cyclohexyl hydroperoxide, methylphenyl hydroperoxide, anthraquinone endoperoxide, hypochlorous acid, alkali metal and alkaline earth metal hypochlorites, tert-butyl hypochlorite, tert-butyl hypobromite, tert-butyl hypoiodite and dinitrogen monoxide.

It is also possible to oxidize iodides or bromides electrochemically to iodine or bromine.

As solvent, preference is given to using water in which ammonia and primary amine, iodides and bromides and the majority of oxidants dissolve sufficiently. Although iodine is soluble only in very small amounts in water, it is readily soluble in the presence of iodides.

However, it can also be advantageous to use mixtures of water and organic solvents which are inert under the reaction conditions. Readily water-soluble and less readily water-soluble solvents are possible here. Readily water-soluble solvents include, for example, ethers such as tetrahydrofuran and dioxane, while less readily soluble solvents include aliphatic, cycloaliphatic and aromatic hydrocarbons such as n-hexane, heptane, cyclohexane and toluene.

The addition of solvents having a low solubility in water generally leads to improved separation of organic and aqueous phases and thus to a simplified work-up of the reaction mixture.

The molar ratio of olefin (I) to ammonia to iodide, iodine or iodide+iodine is preferably 1:1-100:0.001-1.5, particularly preferably 1:1-90:0.01-1.3, very particularly preferably 1:1-80:0.1-1.1. The same molar ratios apply to the ratio of olefin to ammonia to bromide, bromine or bromide+bromine.

The molar ratio of olefin (I) to primary amine (R⁵NH₂) to iodide, iodine or iodide+iodine is preferably 1:1-100:0.001-1.5, particularly preferably 1:1-90:0.01-1.3, very particularly preferably 1:1-80:0.1-1.1.

The same molar ratios apply to the ratio of olefin to primary amine (R⁵NH₂) to bromide, bromine or bromide bromine.

The molar ratio of iodide or iodine to oxidant is preferably 1:1-10, particularly preferably 1:1-4, very particularly preferably 1:1-3.

The molar ratio of olefin (I) to oxidant is preferably 1:1-5, particularly preferably 1:1-3, very particularly preferably 1:2.

The reaction mixture preferably comprises from 30 to 90% by weight of water and from 1 to 30% by weight of organic solvent, particularly preferably from 70 to 80% by weight of water and from 2 to 20% by weight of organic solvent.

In a preferred mode of operation, a surface-active substance is added to the reaction mixture. This effects a significant increase in the aziridine yield.

Suitable surface-active substances are essentially all groups of substances which are mentioned in Ullmanns Encyclopedia of Industrial Chemistry, 6th edition, volume 35, keyword “surfactants”, pages 293 to 368.

These include anionic, cationic, nonionic, amphoteric and anion/cation-surface-active substances (“surfactants”).

Preferred surface-active substances are nonionic surfactants such as polyalkylene glycol alkyl ethers (e.g. Brij®). They are copolymers in which the lipophilic part comprises fatty alcohols and the hydrophilic part comprises short-chain polyalkylene glycols, preferably polyethylene glycols.

As fatty alcohols, preference is given to using the alcohols derived from lauric, palmitic, stearic or oleic acid.

Further examples of suitable nonionic surfactants are:

Tritons® (ethoxylates of 4-(1,1,3,3-tetramethylbutyl)phenol),
Lutensols® (ethoxylated fatty alcohols, alkylphenols or fatty amines),
Tweens® (polyoxyethylene derivatives of sorbitan esters, e.g. polyethoxysorbitan laurate).

The amount of surface-active substances is preferably from 0.01 to 10% by weight, particularly preferably from 0.5 to 5% by weight, very particularly preferably from 1 to 2% by weight, in each case based on the total reaction mixture.

Instead of surface-active substances or in addition to them, the reaction can be carried out in the presence of zeolites and/or other porous inorganic materials. Here too, a significant increase in the aziridine yields can be observed.

Suitable zeolites are essentially all naturally occurring and synthetically obtainable zeolites, i.e. zeolites of the types A, X, Y and L which differ in terms of the pore sizes and the ratio of SiO₂:Al₂O₃ (modulus).

Preference is given to SiO₂-comprising zeolites such as silicalite and zeolites having a high SiO₂ content, i.e. a high modulus, e.g. ZSM-5 zeolite (modulus about 30) and synthetic mordenite (modulus about 10).

The amount of zeolite and/or other porous inorganic materials is preferably from 1 to 20% by weight, particularly preferably from 1 to 10% by weight, very particularly preferably from 1 to 5% by weight, in each case based on the total reaction mixture.

The preparation of the aziridines is preferably carried out at temperatures in the range from 0° C. to 300° C., particularly preferably from 10° C. to 250° C., very particularly preferably from 20° C. to 200° C., for example in the range from 20 to 50° C.

The reaction is preferably carried out at an absolute pressure in the range from 1 bar to 300 bar, particularly preferably from 1 bar to 250 bar, very particularly preferably from 1 to 150 bar, for example in the range from 1 to 10 bar.

The reaction according to the invention can be carried out in one stage, two stages or more than two stages in the liquid phase.

In the case of a single-stage mode of operation, the reactants olefin, ammonia or primary amine, halogen and/or halides are mixed in the presence of an oxidant in water as solvent and, if appropriate, additionally in the presence of an organic solvent, a surface-active substance and/or a suspended or fixed zeolite in a reaction vessel under the reaction conditions indicated for, for example, from 0.1 to 30 hours. The reaction can be carried out batchwise or continuously. In general, separation of the reaction mixture into a liquid aqueous phase and a liquid organic phase is carried out after the reaction.

The liquid organic phase comprises the aziridines formed and possibly unreacted olefins, surface-active substances and organic solvents.

The liquid aqueous phase comprises halogen and halide, ammonia or primary amine and possibly surface-active substances. They can be recirculated to the synthesis stage.

The work-up of the organic phase can be carried out in a manner known per se, e.g. by distillation. Unreacted olefin, organic solvents and surface-active substances can be recirculated to the synthesis stage.

In an advantageous variant of the process of the invention, the iodides or bromides formed in the aziridine synthesis are subsequently oxidized and recirculated to the synthesis stage:

In the two-stage mode of operation, the reactants olefin, ammonia or primary amine and halogen (i.e. bromine or iodine) are, in the first step, mixed without addition of an oxidant in water as solvent and, if appropriate, additionally in the presence of an organic solvent, a surface-active substance and/or a suspended or fixed catalyst, i.e. the above-described zeolites and/or other porous inorganic materials, in a reaction vessel under the reaction conditions indicated for, for example, from 0.1 to 30 hours, The reaction can be carried out batchwise or continuously. After the reaction, the phases are separated. The organic phase is worked up as described for the single-stage mode of operation.

In the second step, the aqueous phase is treated with an oxidant, e.g. an oxidant as described above, or is electrochemically oxidized. Here, iodide or bromide is oxidized to iodine or bromine. The halogen-comprising aqueous phase is then recirculated to the synthesis stage.

### EXAMPLES

The composition of the outputs from the reaction and the yields and selectivities of/to the aziridines were determined by gas chromatography. Brij 35® is the trade name for polyoxyethylene(23) lauryl ether.

Triton® X-100 is a nonionic surfactant comprising ethoxylates of 4-(1,1,3,3-tetramethylbutyl)phenol. Lutensols® are nonionic surfactants based on ethoxylated fatty alcohols, alkylphenois or fatty amines. Tweens® are polyoxyethylene derivatives of sorbitan esters, e.g. polyethoxysorbitan laurate (Tween® 20), polyethoxysorbitan palmitate (Tween® 40) and polyethoxysorbitan oleate (Tween® 80).

### Example 1

Brij 35 (90 mg) and 0.5 mmol iodine (127 mg) were added to 5 ml of a 25% strength by weight aqueous ammonia solution. The reaction was started by addition of 0.5 mmol styrene (57 μl). (The proportion of Brij 35 was thus 2% by weight, and the molarity of iodine and styrene was in each case 0.1 M). After a reaction time of 2 hours at room temperature, the reaction mixture was extracted with diethyl ether. The yield of 2-phenylaziridine was 65% (selectivity >99%).

When the reaction was carried out under identical conditions for 24 hours, the yield was 81% (selectivity=99%).

### Example 2

Brij 35® (180 mg) and 0.5 mmol iodine (127 mg) were added to 5 ml of a 25% strength by weight aqueous ammonia solution. The reaction was started by addition of 0.49 mmol alpha-methylstyrene (65 μl). (The proportion of Brij 35 was thus 4% by weight, and the molarity of iodine and alpha-methylstyrene was in each case 0.1 M). After a reaction time of 2 hours at room temperature, the reaction mixture was extracted with diethyl ether. The yield of 2-methyl-2-phenylaziridine was 76% (selectivity >99%).

### Example 3

a) Brij 35 (180 mg) and 0.5 mmol iodine (127 mg) were added to 5 ml of a 25% strength by weight aqueous ammonia solution. The reaction was started by addition of 0.5 mmol p-chlorostyrene (60 μl). (The proportion of Brij 35 was thus 4% by weight, and the molarity of iodine and p-chlorostyrene was in each case 0.1 M). After a reaction time of 2 hours at room temperature, the reaction mixture was extracted with diethyl ether. The yield of 2-(p-chlorophenyl)aziridine was 60% (selectivity >99%).
b) Under reaction conditions identical to those in Example 3 a), beta-methylstyrene, p-methylstyrene, p-methoxystyrene and m-chlorostyrene were converted into the corresponding aziridines. The following yields were obtained: 1-phenyl-2-methyl-aziridine 30%, 2-(p-methylphenypaziridine 60%, 2-(p-methoxyphenyl)aziridine 56%, 2-(m-chlorophenyl)aziridine 61%.
c) Under identical reaction conditions to those in Example 3 a), styrene was reacted in the presence of a series of uncharged surface-active substances (4% by weight). The yields of 2-phenylaziridine are given in parentheses after the surface-active substances: Triton X-100 (45%), Lutensols, AT25 (49%), FB AT80 (50%), XL 140 (51%), Tween 20 (49%), 40 (51%), 80 (49%).
d) When Example 3c) was carried out in the absence of a surface-active substance, 1% of 2-phenylaziridine was found.

### Example 4

a) When the procedure of example 3c) was repeated in the presence of silicalite (5.5% by weight) in place of surface-active substances, the yield of 2-phenyl-aziridine was 35%.
b) When the procedure of example 4 a) was repeated using MCM-41 (5.5% by weight) in place of silicalite, the yield was 35%.

### Example 5

Brij 35 (180 mg) and 0.5 mmol iodine (127 mg) were added to 5 ml of a 25% strength by weight aqueous ammonia solution. The reaction was started by addition of 0.46 mmol 2-methyl-1-heptene (78 μl). (The proportion of Brij 35 was thus 4% by weight, and the molarity of iodine was 0.1 M and that of 2-methyl-1-heptene was 0.9 M). After a reaction time of 2 hours at 70° C., the reaction mixture was extracted with diethyl ether. The yield of 2-methyl-2-pentylaziridine was 1% (selectivity=98%).

### Example 6

Brij 35® (180 mg), ammonium iodide (73 mg) and 0.5 mmol styrene (57 μl) were added to 5 ml of a 25% strength by weight aqueous ammonia solution. The reaction was started by the addition of a 10-13% strength by weight aqueous sodium hypochlorite solution (400 μl) in portions, with 40 μl being added every 5 minutes. After a reaction time of 2 hours at room temperature, the reaction mixture was extracted with diethyl ether. The yield of 2-phenylaziridine was 74% (selectivity=98%).

### Example 7

Brij 35 (180 mg), ammonium iodide (73 mg) and 0.46 mmol 2-methyl-1-heptene (78 μl) were added to 5 ml of a 25% strength by weight aqueous ammonia solution. The reaction was started by addition of a 10-13% strength by weight aqueous sodium hypochlorite solution (1 ml) in portions. The sodium hypochlorite solution was added in portions of 200 μl every 5 minutes. After a reaction time of 2 hours at room temperature, the reaction mixture was extracted with diethyl ether. The yield of 2-methyl-2-pentylaziridine was 1%.

### Example 8

### Comparative Example

Brij 35 (180 mg) and 0.5 mmol styrene (57 μl) were added to 5 ml of a 25% strength by weight aqueous ammonia solution. The reaction was started by addition of a 10-13% strength by weight aqueous sodium hypochlorite solution (1 ml) in portions, with 200 μl being added every 5 minutes. After a reaction time of 2 hours at room temperature, the reaction mixture was extracted with diethyl ether. The yield of 2-phenylaziridine was <1%.

### Example 9

Brij 35® (180 mg), ammonium iodide (50 mol %, 37 mg) and 0.5 mmol styrene (57 μl) were added to 5 ml of a 25% strength aqueous ammonia solution. The reaction was started by addition of a 10-13% strength by weight aqueous solution of sodium hypochlorite (400 μl) in portions (40 μl/5 min.). After a reaction time of two hours at room temperature, the reaction mixture was extracted with diethyl ether. The yield of 2-phenylaziridine was 60%.

### Example 10

Brij 35® (180 mg), ammonium iodide (29 mg) and 0.5 mmol of styrene (57 μl) were added to 5 ml of a 1.3 molar aqueous ammonia solution. The reaction was started by addition of a 10-13% strength by weight aqueous solution of sodium hypochlorite (400 μl) in portions, with 40 μl being added every 5 minutes. After a reaction time of 2 hours at room temperature, the reaction mixture was extracted with diethyl ether. The yield of 2-phenylaziridine was 90% (selectivity: 99%).

### Example 11

Brij 35® (180 mg), ammonium iodide (29 mg) and 0.5 mmol (for amount, see below) of a substituted styrene derivative were added to 5 ml of a 1.3 molar aqueous ammonia solution. The reaction was started by addition of a 10-13% strength by weight aqueous solution of sodium hypochlorite (400 μl) in portions, with 40 μl being added every 5 minutes. After a reaction time of 2 hours at room temperature, the reaction mixture was extracted with diethyl ether. The yields of the individual substituted 2-phenylaziridines were:

74% of 2-(p-chlorophenyl)aziridine (selectivity: 99%) from p-chlorostyrene (60 μl)
92% of 2-methyl-2-phenylaziridine (selectivity: 99%) from alpha-methylstyrene (65 μl)
56% of 2-methyl-3-phenylaziridine (selectivity: 98%) from beta-methylstyrene(65 μl )

### Example 12

Brij 35® (90 mg) and 0.5 mmol of iodine (127 mg) were added to 5 ml of a 1 molar aqueous methylamine solution. The reaction was started by addition of 0.5 mmol of styrene (57 μl). After a reaction time of 2 hours at room temperature, the reaction mixture was extracted with diethyl ether. The yield of 1-methyl-2-phenylaziridine was 43% (selectivity: 84%).

When the reaction was carried out for 5 hours under the same conditions, the yield was 64% (selectivity: 86%).

### Example 13

Brij 35® (90 mg) and 0.5 mmol of iodine (127 mg) were added to 5 ml of a 0.6 molar aqueous ethylamine solution. The reaction was started by addition of 0.5 mmol of styrene (57 μl). After a reaction time of 2 hours at room temperature, the reaction mixture was extracted with diethyl ether. The yield of 1-ethyl-2-phenylaziridine was 28% (selectivity: 93%).

### Example 14

Brij 35® (90 mg) and 0.5 mmol of iodine (127 mg) were added to 5 ml of a 1 molar aqueous methylamine solution. The reaction was started by addition of 0.5 mmol of p-chlorostyrene (60 μl). After a reaction time of 2 hours at room temperature, the reaction mixture was extracted with diethyl ether. The yield of 1-methyl-2-(p-chlorophenyl)aziridine was 39% (selectivity: 93%).

### Example 15

Brij 35® (90 mg) and 0.2 mmol of iodine (254 mg) were added to 5 ml of a 1 molar aqueous methylamine solution. The reaction was started by addition of 0.5 mmol of styrene (57 μl). After a reaction time of 2 hours at room temperature, the reaction mixture was extracted with diethyl ether. The yield of 1-methyl-2-phenylaziridine was 68% (selectivity: 83%).

### Example 16

Brij 35® (90 mg) and 0.1 mmol of iodine (127 mg) were added to 5 ml of a 1 molar aqueous methylamine solution. The reaction was started by addition of 0.48 mmol of 2-methyl-1-heptene (57 μl). After a reaction time of 2 hours at room temperature, the reaction mixture was extracted with diethyl ether. The yield of 1-methyl-2-phenylaziridine was 3% (selectivity: 83%).

### Example 17

Brij 35® (90 mg), ammonium iodide (73 mg) and 0.5 mmol of styrene (57 μl) were added to 5 ml of a 1 molar aqueous methylamine solution. The reaction was started by addition of a 10-13% strength by weight aqueous sodium hypochlorite solution (400 μl) in portions, with 40 μl being added every 5 minutes. After a reaction time of 2 hours at room temperature, the reaction mixture was extracted with diethyl ether. The yield of 1-methyl-2-phenylaziridine was 39% (selectivity: 95%).

### Example 18

Brij 35® (90 mg), ammonium iodide (73 mg) and 0.5 mmol of styrene (57 μl) were added to 5 ml of a 1 molar aqueous methylamine solution. The reaction was started by addition of a 10-13% strength by weight aqueous sodium hypochlorite solution (1 ml) in portions, with 100 μl being added every 5 minutes. After a reaction time of 2 hours at room temperature, the reaction mixture was extracted with diethyl ether. The yield of 1-methyl-2-phenylaziridine was 73% (selectivity: 97%).

### Example 19

Brij 35® (90 mg), ammonium iodide (73 mg) and 0.5 mmol of styrene (57 μl) were added to 5 ml of a 0.5 molar aqueous methylamine solution. The reaction was started by addition of a 10-13% strength by weight aqueous sodium hypochlorite solution (1 ml) in portions, with 100 μl being added every 5 minutes. After a reaction time of 2 hours at room temperature, the reaction mixture was extracted with diethyl ether. The yield of 1-methyl-2-phenylaziridine was 52% (selectivity: 95%).

### Example 20

Brij 35® (90 mg), ammonium iodide (73 mg) and 0.5 mmol of styrene (57 μl) were added to 5 ml of a 0.6 molar aqueous ethylamine solution. The reaction was started by addition of a 10-13% strength by weight aqueous sodium hypochlorite solution (1 ml) in portions, with 100 μl being added every 5 minutes. After a reaction time of 2 hours at room temperature, the reaction mixture was extracted with diethyl ether. The yield of 1-ethyl-2-phenylaziridine was 55% (selectivity: 96%).

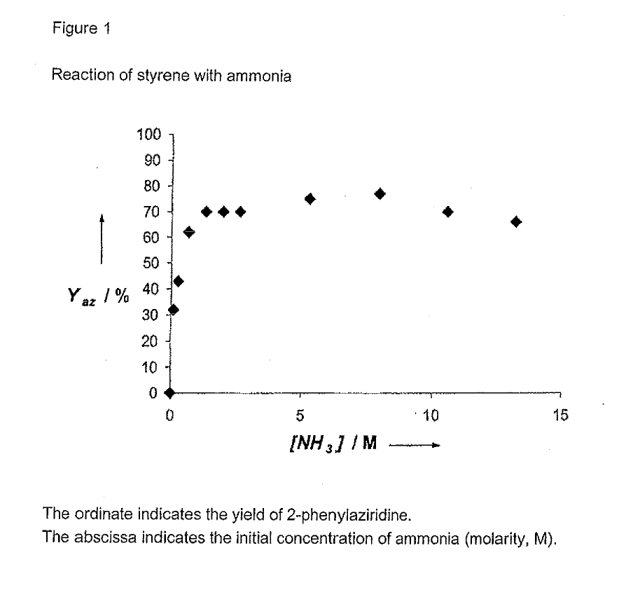

FIG. 1 below shows, for the example of the reaction according to the invention of styrene with ammonia (NH₃), the dependence of the yield of 2-phenylaziridine on the initial ammonia concentration. The conditions of the experiments corresponded to those of example 6, except that the NH₃ concentration was varied. As can be seen, the preferred ammonia concentration range found is from ≦1.2 to 15 molar. In contrast, the preferred primary amine concentration range found in reactions according to the invention with primary amines (R⁵NH₂) is surprisingly from >0.5 to ≦1.1 molar. Also compare examples 6 and 19.

1.-28. (canceled)

29. A process for preparing an N-unsubstituted aziridine of the formula II 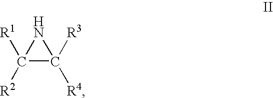
which comprises reacting an olefin of the formula I 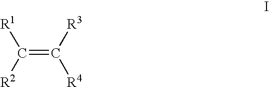
where R¹ to R⁴ are each, independently of one another, hydrogen, a linear or branched alkyl radical having from 1 to 16 carbon atoms, a hydroxyalkyl radical having from 1 to 4 carbon atoms, a cycloalkyl radical having from 5 to 7 carbon atoms, a benzyl or phenyl radical which in each case may be substituted in the o, m or p position of the phenyl radical by methoxy, hydroxy, chlorine or alkyl radicals having from 1 to 4 carbon atoms and the radical R¹ or R² together with the radical R³ or R⁴ may be closed to form a 5- to 12-membered ring or the radicals R¹ and R² may be closed to form a 5- to 12-membered ring,
with ammonia in the presence of iodine or bromine and carrying out the aziridine synthesis in the presence of a surface-active substance.

30. The process according to claim 29, which comprises reacting an olefin of the formula I with ammonia in the presence of an iodide and an oxidant which is able to oxidize the iodide to iodine.

31. The process according to in claim 29, which comprises reacting an olefin of the formula I with ammonia in the presence of a bromide and an oxidant which is able to oxidize the bromide to bromine.

32. The process according to claim 29, wherein the concentration of the ammonia in the reaction mixture at the beginning of the reaction is greater than or equal to 1.2 molar (≦1.2 M).

33. A process for preparing an N-substituted aziridine of the formula III 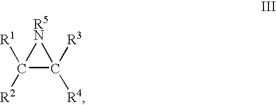
which comprises reacting an olefin of the formula I 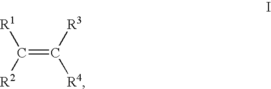
where R¹ to R⁴ are each, independently of one another, hydrogen and R¹ to R⁵ are each, independently of one another, a linear or branched alkyl radical having from 1 to 16 carbon atoms, a hydroxyalkyl radical having from 1 to 4 carbon atoms, a cycloalkyl radical having from 5 to 7 carbon atoms, a benzyl radical or phenyl radical which may in each case be substituted in the o, m or p position of the phenyl radical by methoxy, hydroxy, chlorine or alkyl radicals having from 1 to 4 carbon atoms and the radical R¹ or R² may be closed with the radical R³ or R⁴ to form a 5- to 12-membered ring or the radicals R¹ and R² may be closed to form a 5- to 12-membered ring,
with a primary amine of the formula R⁵NH₂ in the presence of iodine or bromine, where the concentration of the primary amine (R⁵NH₂) in the reaction mixture is less than or equal to 1.1 molar (≦1.1 M), and carrying out the aziridine synthesis in the presence of a surface-active substance.

34. The process according to claim 33, which comprises reacting an olefin of the formula I with a primary amine of the formula R⁵NH₂ in the presence of an iodide and an oxidant which is able to oxidize the iodide to iodine, where the concentration of the primary amine (R⁵NH₂) in the reaction mixture is less than or equal to 1.1 molar (≦1.1 M).

35. The process according to claim 33, which comprises reacting an olefin of the formula I with a primary amine of the formula R⁵NH₂ in the presence of a bromide and an oxidant which is able to oxidize the bromide to bromine, where the concentration of the primary amine (R⁵NH₂) in the reaction mixture is less than or equal to 1.1 molar (≦1.1 M).

36. The process according to claim 29, wherein the concentration of the primary amine (R⁵NH₂) in the reaction mixture at the beginning of the reaction is greater than 0.5 molar (>0.5 M).

37. The process according to claim 30, wherein the iodide is an alkali metal, alkaline earth metal, ammonium or tetraalkylammonium iodide or a mixture thereof.

38. The process according to claim 30, wherein the bromide is an alkali metal, alkaline earth metal, ammonium or tetraalkylammonium bromide or a mixture thereof.

39. The process according to claim 30, wherein the iodide is a mixture of iodide and iodine.

40. The process according to claim 31, wherein the bromide is a mixture of bromide and bromine.

41. The process according to claim 30, wherein the oxidant is oxygen, hydrogen peroxide, cumene hydroperoxide, methylphenyl hydroperoxide, anthraquinone endoperoxide, hypochlorous acid, tert-butyl hypochlorite, tert-butyl hypobromite, tert-butyl hypoiodite, dinitrogen monoxide, an alkali metal hypochlorite or an alkaline earth metal hypochlorite.

42. The process according to claim 30, wherein the oxidant is an anode.

43. The process according to claim 30, wherein the aziridine synthesis is carried out in the presence of a surface-active substance.

44. The process according to claim 29, wherein the aziridine synthesis is carried out in the presence of a nonionic surface-active substance.

45. The process according to claim 44, wherein the nonionic surface-active substance is polyalkylene glycol alkyl ether, an ethoxylated fatty alcohol, an ethoxylated alkylphenol or an ethoxylated fatty amine.

46. The process according to claim 29, wherein the reaction is carried out in the presence of water.

47. The process according to claim 29, wherein the reaction is carried out in the presence of water and an organic solvent.

48. The process according to claim 47, wherein the organic solvent is an ether, an aliphatic hydrocarbon, a cycloaliphatic hydrocarbon or aromatic hydrocarbon.

49. The process according to claim 29, wherein the aziridine synthesis is carried out in the presence of a zeolite.

50. The process according to claim 29, wherein the molar ratio of olefin (I) to ammonia to halide, halogen or halide+halogen is 1:1-100:0.001-1.5 and the molar ratio of olefin (I) to primary amine (R⁵NH₂) to halide, halogen or halide+halogen is 1:1-100:0.001-1.5.

51. The process according to claim 29, wherein the preparation of the aziridine is carried out at a temperature in the range from 0° C. to 300° C.

52. The process according to claim 29, wherein the preparation of the aziridine is carried out at an absolute pressure in the range from 1 to 300 bar.

53. The process according to claim 29, wherein the preparation of the aziridine is carried out in two substeps, with the reaction of the olefin (I) with ammonia or primary amine (R⁵NH₂) in the presence of iodine or bromine being carried out in the first substep and the halide formed in the first substep being reoxidized to the corresponding halogen in the second substep and recirculated to the first substep.

54. The process according to claim 29 for preparing aziridine or N-methylaziridine or N-ethylaziridine, wherein ethylene is reacted with ammonia or monomethylamine or monoethylamine.

55. The process according to claim 29 for preparing 2-methylaziridine or 1,2-dimethylaziridine or 1-ethyl-2-methylaziridine, wherein propylene is reacted with ammonia or monomethylamine or monoethylamine.

56. The process according to claim 29 for preparing 2,2-dimethylaziridine or 1,2,2-trimethylaziridine or 1-ethyl-2,2-dimethylaziridine, wherein isobutene is reacted with ammonia or monomethylamine or monoethylamine.
